(19) United States
(12) Patent Application Publication
(10) Pub. No.: US 2017/0196846 A1
(43) Pub. Date: Jul. 13, 2017

(54) METHODS FOR TREATING ATTENTION DEFICIT HYPERACTIVITY DISORDER WITH METHYLPHENIDATE

(71) Applicant: Rhodes Pharmaceuticals, L.P., Coventry, RI (US)

(72) Inventor: Akwete L. Adjei, East Greenwich, RI (US)

(21) Appl. No.: 15/316,651

(22) PCT Filed: Jun. 5, 2015
(86) PCT No.: PCT/US15/34466

§ 371 Date: Dec. 6, 2016

Related U.S. Application Data
(60) Provisional application No. 62/008,890, filed on Jun. 6, 2014.
(60) Provisional application No. 62/053,548, filed on Sep. 22, 2014.
(60) Provisional application No. 62/149,216, filed on Apr. 17, 2015.

(51) Int. Cl. A61K 31/4458

(52) U.S. Cl. CPC A61K 31/4458

(57) ABSTRACT

The invention relates to methods of treating attention deficit hyperactivity disorder in patients in need thereof, and methods for selecting therapeutic dosage amounts to achieve the desired efficacy more quickly. More specifically, the methods of the present invention provide for methylphenidate treatment regimens wherein the efficacious dosage amounts are selected based upon the body weight of the patient to be treated.

### FIELD OF THE INVENTION

The invention relates to methods of treating attention deficit hyperactivity disorder in patients in need thereof, and methods for selecting therapeutically effective dosage amounts to achieve the desired efficacy more quickly.

### BACKGROUND OF THE INVENTION

Attention Deficit Hyperactivity Disorder (ADHD) is a common neurobehavioral disorder of childhood, occurring in an estimated 7% to 10% of school-aged children and persisting into adolescence in approximately 70% of patients. Children and adolescents with ADHD share many of the phenotypic characteristics of the disorder, although adolescents may manifest more symptoms of inattention relative to hyperactivity and impulsivity. Stimulant medications are widely recommended among first-line therapies for ADHD, and their safety and efficacy are well established.

Prescription stimulants have a paradoxically calming and “focusing” effect on individuals with ADHD. They are prescribed to patients for daily use and come in the form of tablets or capsules of varying dosages. Treatment of ADHD with stimulants, often in conjunction with psychotherapy, helps to improve ADHD symptoms along with the patient's self-esteem, thinking ability, and social and family interactions. The most commonly used measure purported to assess ADHD-related impairment, and improvement of symptoms following treatment, especially in children, is the Swanson, Kotkin, Agler, M-Flynn, and Pelham Scale (SKAMP). The SKAMP specifically assesses context-bound behaviors critical to success in school settings, which is often the most problematic domain of functioning for children with ADHD. The 10 SKAMP items were initially developed as modifications of target behaviors used in specialized classroom management systems. Hence, the items are framed as “difficulties” and “problems” that would be expected to reflect ADHD-related classroom impairment, including the performance of academic tasks, following class rules, and interacting with peers and adults in the classroom. However, additional evaluation techniques are utilized to measure ADHD symptoms in adolescents and adults primarily due to the lack of a classroom setting as required by the SKAMP.

These evaluation techniques include the assessment criteria provided in the American Psychiatric Association's Diagnostic and Statistical Manual of Mental Disorders (DSM). One assessment tool is a 55-question assessment that reviews symptoms of ADHD according to the DSM-IV criteria, and possible inclusion of behavioral criteria identified in DSM-V. Another commonly used tool is the ADHD Rating Scale-IV (ADHD-RS-IV), comprising 18 total items, 9 directed to inattention and 9 to hyperactivity, and all based on the DSM-IV criteria. The total score obtained from the ADHD-RS-IV is frequently used to not only diagnose patients with ADHD, but also to measure effectiveness of treatment of ADHD in adults and children.

The most commonly prescribed stimulant is methylphenidate (MPH). Methylphenidate (2-piperidineacetic acid, α-phenyl-methyl ester) is a piperidine derivative that is structurally related to amphetamine and is the psychostimulant. Generally, MPH appears to have a higher incidence of positive effects and a lower incidence of adverse effects than other psychostimulants. Many formulations of MPH are commercially available, including immediate release (RITALIN IR®, METHYLIN®) and sustained, extended, or long acting formulations (RITALIN SR®, RITALIN LA®, DAYTRANA®, CONCERTA®, METADATE CD®, METADATE CD®, METADATE ER®, METHYLIN ER® QILLIVANT XR®). Although a large number of patients treated with methylphenidate have some positive therapeutic response, clinicians continue to struggle with selecting the appropriate formulation and optimal dose of methylphenidate to prescribe to a patient due to the high variability in therapeutic response. This variability seems to be exacerbated by the finding that in general, drug clearance in children appears to be an important parameter in determining pediatric dosing. These findings suggest that pediatric dosing should not be determined by simply applying weight-based calculations to an adult dose (W. Rodriguez et al., Pediatrics Vol. 121 No. 3, Mar. 1, 2008, pp. 530-539)

The typical prescribing information for psychostimulants, including methylphenidate, indicates that the prescribing physician should initiate therapy at a relatively low dose and then adjust the dose upwards at weekly intervals, depending on the patient tolerability and the degree of efficacy. This titration period can be weeks or even months, resulting in patients receiving suboptimal treatment, inadequate resolution of symptoms, patient frustration, and even patients abandoning treatment all together.

There is a need in the art for methods of treating ADHD in patients with psychostimulants that enable a physician to initiate treatment at a dose that provides patients with rapid relief from the symptoms of ADHD, while decreasing the duration of the titration period or eliminating it altogether.

### BRIEF SUMMARY OF THE INVENTION

In one aspect, the invention provides a method of treating attention deficit hyperactivity disorder (ADHD) in a patient comprising determining the body weight of the patient in need of treatment, selecting a dosage form of a controlled-release formulation of methylphenidate to be administered to the patient, and administering the selected dosage form to the patient, wherein the selected dosage form contains a therapeutically effective amount of methylphenidate, and the selected dosage form is determined by the amount of methylphenidate contained in the dosage form and the body weight of the patient in need of treatment.

In another aspect, the invention provides a method of reducing or eliminating a titration period of a treatment regimen for treating attention deficit hyperactivity disorder (ADHD) in a patient with methylphenidate comprising determining the body weight of the patient in need of treatment, selecting a dosage form of a controlled-release formulation of methylphenidate to be administered to the patient, and administering the selected dosage form to the patient, wherein the selected dosage form contains a therapeutically effective amount of methylphenidate, and the selected dosage form is determined by the amount of methylphenidate contained in the dosage form and the body weight of the patient in need of treatment.

### BRIEF DESCRIPTION OF THE DRAWINGS

FIG. 1 demonstrates the mean simulated concentration-time profiles for Study Formulation dose strengths between 10 mg and 80 mg.

FIG. 2 demonstrates the simulated mean change from baseline ADHD total score for a range of body weights and a range of Study Formulation fixed dosage strengths.

FIG. 3 demonstrates a correlation between patient body weight and the amount of methylphenidate in the dosage forms selected and administered in the methods of the present invention that achieve at least an 18 point decrease in total ADHD Score.

### DETAILED DESCRIPTION OF THE INVENTION

The methods of the instant invention relate to treating attention deficit hyperactivity disorder in a patient comprising administering a therapeutically effective amount of methylphenidate to the patient. Although the treatment of ADHD with methylphenidate is well known, problems still exist with the current methodologies. Perhaps one of the most difficult aspects of treating ADHD, especially in children, is determining what dose and formulation of methylphenidate to prescribe to the patient to achieve the desired efficacy early in treatment without overdosing the patient but also without delaying efficacy by under dosing. Although there are a number of different doses and formulations of methylphenidate available commercially, none of them provide dosing regimens for both children and adults that begin treatment with a dosage amount that is likely to be a therapeutically effective amount. Instead, the recommended dosing regimen begins with a titration regimen, starting the patient at a low dose for seven days, followed by weekly upward adjustments until the optimal response is reached. Alternatively, if a patient is changing from one methylphenidate formulation to another, a conversion calculation is used, though often yet another titration period is required to achieve the desired efficacy. In addition, many of the available formulations that are prescribed using a titration regiment are not available in more than a few dosage strengths, leaving the prescribing clinician with a difficult choice of either under- or over-dosing in some situations simply because the optimal dosage amount for that patient is not commercially available.

Extensive studies have been conducted to understand the pharmacodynamics (pd) and pharmacokinetic (pk) profiles of MPH formulations in children and adults. However, it has been consistently found that plasma MPH levels are subject to a considerable degree of inter-patient variability, and each formulation of MPH appears to provide a different pk/pd profile and a slightly different therapeutic effect. In treating ADHD with MPH, it is desired that the onset of action occurs from about 0.5 to about 4 hours, and preferably from about 0.5 to about 2 hours after the oral dosage form is administered. Further, it is desired that the dosage form no longer provides effective plasma levels of methylphenidate from about 8 to about 12 hours after oral administration of the dose. In this manner, the dose of methylphenidate can be administered to a child in the morning before school begins, providing the desired effect at the start of the school day, with the pharmacologic action of the drug not waning until after the school day ends, but preferably before dinner so that the drug does not have the side effect of acting as an appetite suppressant or affecting the patient's ability to sleep.

The desired clinical effect described above is dependent on reaching and maintaining plasma drug concentrations within an appropriate therapeutic range at the appropriate time following drug administration. However, rapidly achieving that effect continues to be a struggle for physicians and patients. Even though the pharmacokinetics and pharmacodynamics of many MPH formulations have been studied for over 50 years, inter-patient variability has prevented the identification of a measurable and accurate surrogate marker for determining a therapeutically effective dose for each patient, such as age, weight, height or gender. Instead, the prescribing information for the commercially available formulations of MPH recommend initiating treatment at low doses and titrating upward at weekly intervals until the desired effect is achieved. Although some patients may benefit from the initial low doses, many patients require multiple weeks of titration before reaching the desired therapeutic response. This time period of titration from initial dose to the desired efficacious dose is referred to herein as the “titration period.”

The inventors have surprisingly found that certain formulations of methylphenidate can be used to treat ADHD in both children and adults, wherein a therapeutically effective amount can be administered at the onset of treatment, or shortly thereafter, thus reducing or eliminating the titration period. The method of treatment comprises selecting a therapeutically effective amount of methylphenidate based on the body weight of the patient. In one embodiment, the dosage form is administered once a day. Considering the desired clinical effects, the dosage form can be administered to the patient every day, only on work or school days, or only on days wherein the patient's ADHD symptoms interfere with the patient's daily life. Thus, in one embodiment, the patient is administered the selected dosage form for at least two consecutive days or at least three consecutive days. In another embodiment, the patient is administered the selected dosage form for at least five consecutive days, at least ten consecutive days, or at least thirty consecutive days.

The therapeutically effective amount selected based on the patient's body weight can, in one embodiment, be an initial dose of methylphenidate administered to the patient. In another embodiment, the therapeutically effective amount selected based on the patient's body weight can be a subsequent dose of methylphenidate. In certain embodiments, the initial dose of methylphenidate administered to the patient is less than the therapeutically effective amount as selected based on the patient's body weight, wherein one or more of the subsequent dose or doses of methylphenidate are selected based upon the patient's body weight. In another embodiment, the therapeutically effective amount selected based on the patient's body weight is an initial dose and a subsequent dose. In one embodiment, the titration period is eliminated wherein in another embodiment, the titration period is reduced.

In one embodiment, the patient does not suffer significantly from any co-morbidity such as diabetes, obesity, kidney or liver disease.

It is known that clearance of methylphenidate is an important factor in reaching and maintaining therapeutically effective levels of MPH in a patient. However, it is also known that drug clearance in children and adults does not always correlate well with their total body weight. Not being bound by any particular theory, the formulations of methylphenidate employed in the methods of the present invention provide biphasic release profiles seemingly similar to other commercially available methylphenidate formulations. However, the formulations employed in the instant invention demonstrate a good correlation between patient body weight and therapeutically effective dose. This correlation appears to be possible since the formulations employed in the instant invention generally provide a lower fluctuation index and high predictability of multiple pharmacokinetic/pharmacodynamic (PK/PD) parameters as compared to other methylphenidate formulations.

Fluctuation index (FI) is an important pharmacokinetic parameter for assessing variability in plasma concentrations at steady state. The fluctuation index is the peak-to-trough plasma-concentration variation that can be calculated by various methods known in the art. One convenient method is to calculate the ratio of the maximum plasma concentration C₍ₘₐₓ₎ to the trough or minimum concentration C₍ₘᵢₙ₎. However, more accurate results can be obtained when also taking into account the average plasma concentration C₍ₐᵥₑ₎ as shown in the following equation:

FI=(C₍ₘₐₓ₎−C₍ₘᵢₙ₎)/C₍ₐᵥₑ₎.

In general, a lower fluctuation index is associated with less variability in plasma concentrations at steady state. Serial doses of immediate release MPH and pulsatile release formulations of MPH demonstrate multiple peaks and valleys in plasma concentrations. The FI can be calculated for each of these “peaks”, and the lower the FI, the higher the predictability of the drug exposure to the patient. For example, the plasma profile of a multilayer formulation with an immediate release portion of methylphenidate exterior to a delayed release portion of methylphenidate will demonstrate two plasma profile peaks. Generally, the first peak corresponds to the immediate release portion and the second peak generally corresponds to the delayed release portion. Determining FI for each of these peaks can be very useful in evaluating extended or sustained release pharmaceutical formulations as well as dosing regimens intended to have long acting effects.

Methylphenidate formulations that are not those employed in the instant invention appear to demonstrate inconsistent or enlarging fluctuation indices over time, especially in the plasma concentration peaks that correlate to the delayed release portions of a pulsatile release formulation. Inconsistencies in FI tend to parallel unpredictability also seen in other PK/PD parameters of these formulations. By contrast, the formulations employed in the methods of the instant invention demonstrate a relatively low FI and relatively high reproducibility and predictability of PK/PD parameters in adult, adolescent, and pediatric studies. The relatively lower fluctuation index and the reproducibility of key PK/PD parameters such as absorption, distribution, elimination and clearance, allow for good correlation of a therapeutically effective dosage amount of MPH with the patient's body weight. This correlation permits a treatment method that reduces or eliminates the need for a titration period, since a therapeutically effective dosage of MPH can be readily determined by the body weight of the patient and the patient can begin treatment with a therapeutically efficacious dosage amount. In one embodiment, the FI of the second peak of formulations employed in the present invention is less than 0.3. In another embodiment, the FI of the second peak is less than 0.2. In a further embodiment, the FI of the second peak is less than 0.1. In certain embodiments, the second peak is associated with a delayed release portion of a pulsatile release formulation of methylphenidate.

As used herein, the term “methylphenidate” includes the active ingredient which is the racemic mixture of two optical isomers d-threo-methylphenidate and 1-threo-methylphenidate, and includes pharmaceutically acceptable salts of the active ingredient. In certain embodiments, the methylphenidate is a hydrochloride salt of methylphenidate.

As used herein, the phrase “immediate release” is the release of an active pharmaceutical ingredient (e.g., MPH) from a pharmaceutical formulation where the rate of release of the active pharmaceutical ingredient from the pharmaceutical formulation is not retarded by means of a controlled release matrix or other such means and where the components of the pharmaceutical formulation are designed such that, upon ingestion, maximum exposure of said active pharmaceutical ingredient to body tissues occurs in the minimum period of time. As described herein, an “immediate release” MPH component preferably releases in less than 1 hour, e.g., as soon as about 45 minutes or as soon as about 30 minutes following administration.

As used herein, the phrase “delayed release” refers to compositions which are characterized by having at least one active ingredient (e.g. MPH) wherein the release of the active ingredient is retarded by means of a controlled-release matrix, enteric coating, or other such means that provides a period of time after ingestion that little or no active ingredient is exposed to tissues in the body. As described herein, the “delayed release” portions of the MPH compositions preferably retard release of MPH in the delayed release portion of the MPH formulation for at least 15 minutes after ingestion. In one embodiment, the MPH in the delayed release portion is retarded for the period of time until after the formulation passes through the stomach of the patient.

The release profiles of the MPH formulations may be assessed via in vitro dissolution using techniques known to those of skill in the art (e.g., USP basket method, Paddle Method, channel flow method, or other methods known in the literature). The release profile can be assessed in vivo (e.g., for bioavailability determinations), using plasma concentrations to assess maximum plasma concentration (Cₘₐₓ) and area under the curve (AUC). Such assays are well known to those of skill in the art.

The methylphenidate formulations employed in the methods of the present invention are controlled-release formulations. In certain embodiments, the controlled-release MPH formulations used herein are oral dosage form comprising both an immediate release portion and a delayed release portion of MPH. Suitable formulations as used herein include those described in U.S. Pat. No. 6,419,960, U.S. Pat. No. 7,083,808, U.S. Pat. No. 7,247,318, U.S. Pat. No. 7,438,930, and U.S. Pat. No. 8,580,310, such descriptions being incorporated herein in their entirety. In one embodiment, the formulation is an oral controlled release formulation of methylphenidate hydrochloride which provides a rapid onset of therapeutic effect and a rapid drop in plasma concentration after a prolonged period of therapeutic effect, comprising a plurality of substrates comprising a portion of an effective dose of methylphenidate hydrochloride in immediate release form, a hydrophobic material comprising an acrylic polymer coated onto the surface of said substrates in an amount sufficient to retard the release of said drug, an enteric coating applied over said hydrophobic coating in an amount sufficient to substantially delay the release of said drug from said substrate until after said formulation passes through the stomach, wherein said enteric coating is derived from an aqueous dispersion comprising an acrylic/methacrylic copolymer, a plasticizer and a glidant, the formulation further comprising the remaining portion of an effective dose of methylphenidate hydrochloride in immediate release form, and wherein the formulation provides a time to maximum plasma concentration of said methylphenidate hydrochloride at about 0.5 to about 4 hours after oral administration. In another embodiment, the formulation is an oral controlled-release formulation which provides a rapid onset of therapeutic effect and a rapid drop in plasma concentration after a prolonged period of therapeutic effect, comprising a plurality of substrates comprising a portion of an effective dose of a drug in immediate release form, a hydrophobic material coated onto the surface of said substrates in an amount sufficient to retard the release of said drug, an enteric coating applied over said hydrophobic coating in an amount sufficient to substantially delay the release of said drug from said substrate until after said formulation passes through the stomach, the formulation further comprising the remaining portion of an effective dose of the drug in immediate release form; wherein the oral dosage form provides a time to maximum plasma concentration of the drug at about 0.5 to about 4 hours after oral administration and wherein the duration of effect provided by the drug contained in the formulation falls below effective plasma concentrations at about 8 to about 12 hours after oral administration. In another embodiment, the formulation of methylphenidate is an oral controlled release formulation comprising a plurality of multi-layer release beads, each bead comprising (i) an immediate release core comprising a first portion of an effective dose of methylphenidate or a pharmaceutically acceptable salt thereof coated over an inert pharmaceutically acceptable bead; (ii) a controlled release layer coated over the core, the controlled release layer comprising a hydrophobic material in an amount sufficient to provide a controlled release of the first portion of the methylphenidate over a predetermined period of time, the hydrophobic material selected from the group consisting of an alkylcellulose, an acrylic polymer and mixtures thereof; (iii) a release delaying layer coated over the controlled release layer, the release delaying layer comprising a pH-dependent polymer in an amount sufficient to delay release of the first portion of the effective dose of methylphenidate or the pharmaceutically acceptable salt thereof until after the formulation passes through the stomach; and (iv) an outer layer coated over the release delaying layer, the outer layer comprising a second portion of the effective dose of methylphenidate or the pharmaceutically acceptable salt thereof; wherein the formulation provides: (a) a maximum plasma concentration of methylphenidate at about 0.5 to about 4 hours after an oral administration to a human patient, (b) a plasma concentration of methylphenidate which does not differ by more than 20% during a measuring interval, wherein the measuring interval is from about 2 hours to about 6 hours, (c) the plasma concentration of methylphenidate which is below effective plasma concentrations in said human patient at about 8 to 12 hours after the oral administration, and (d) an in-vitro dissolution at 0.25, 1, 4, 8 and 12 hours of 0-45%, 5-50%, 40-90%, not less than 60% and not less than 80% methylphenidate dissolved, respectively.

In certain embodiments, the dosage forms of MPH employed in the invention are formulations that comprise a delayed release portion of MPH and an immediate release MPH. Generally the delayed release portion is at least 50% of the total MPH in the dosage form. Preferably, the delayed release portion of MPH is between about 55% to about 65%. The immediate release portion of MPH in the dosage form is less than 55% of the total MPH in the dosage form. Preferably, the immediate release portion of MPH is between about 35% to about 45%.

The immediate release and the delayed release portions of the MPH formulations can be prepared in accordance with the methods generally known to the skilled artisan. In certain embodiments, the formulations are bead-in-capsule formulations. In one embodiment, the immediate release and the delayed release portions of MPH are on separate beads. In another embodiment, the immediate release and the delayed release portions of MPH are contained within the same bead. In another embodiment, the MPH formulations are multilyer bead-in-capsule formulations wherein the delayed release portion of MPH is in the interior of the beads, the immediate release portion of MPH is on the exterior of the beads, and the portions are separated by a means for providing delayed release of the interior portion of MPH. In a further embodiment, the delayed release portion of MPH in the formulation is not released until after the formulation passes through the stomach of the patient.

As used herein, the phrase “therapeutically effective amount” is an amount that partially or completely provides the desired therapeutic effect. For purposes of the present invention, a therapeutically effective amount of methylphenidate is an amount that provides a decrease in symptoms of ADHD. In one embodiment, the reduction in ADHD symptoms is a change from baseline of at least 18 in the total ADHD-RS-IV score assessment.

In one embodiment, the therapeutically effective amount of methylphenidate is in a dosage form comprising a total amount of methylphenidate selected from 10 mg, 15 mg, 20 mg, 30 mg, 40 mg, 50 mg, 60 mg, and 80 mg.

In another embodiment, the amount of methylphenidate in the selected dosage form is the amount of methylphenidate that corresponds to the body weight of the patient according to the following table:

| Patient Body | Amount of |
|---|---|
| Weight (lb) | Methylphenidate (mg) |
| ≦40 | 10-15 |
| 40-50 | 20 |
| 50-70 | 30 |
| 70-90 | 40 |
| 90-110 | 50 |
| 110-130 | 60 |
| ≧130 | 80 |

The dosage amount selected for a patient that has a weight that is borderline, meaning the weight is indicated in more than one row in the table above, such as a patient weighing 40, 50, 70, 90, 110 or 130 pounds, will be selected using not only the table above but also using the general knowledge of the prescribing physician. To determine the appropriate amount of methylphenidate to be selected for patients of these weights, those skilled in the art will readily recognize the additional factors that can be considered by the prescribing physician when selecting the dosage amount appropriate for such patients. Such factors can include whether the patient is methylphenidate naïve or methylphenidate experienced, the age, gender or ethnicity of the patient, the patient's lean body mass compositions, concomitant medications, physical activity level of the patient, the severity of the patient's symptoms, and whether the patient suffers from any co-morbidities. In one embodiment, the dosage amount selected and administered is the higher dosage amount indicated for the patient with a borderline weight. In another embodiment, the dosage amount selected and administered is the lower dosage amount indicated for the patient with a borderline weight. In another embodiment, the dosage amount is selected from the following table:

| Patient Body | | Dose of |
|---|---|---|
| Weight (lb) | | Methylphenidate (mg) |
| i) | ≦40 | 10 to 15 |
| ii) | 40-50 | 15 to 20 |
| iii) | 50-70 | 20 to 30 |
| iv) | 70-90 | 30 to 40 |
| v) | 90-110 | 40 to 50 |
| vi) | 110-130 | 50 to 60 |
| vii) | ≧130 | 60 to 80 |

In some embodiments, the dosage amount of methylphenidate is not more than 60 mg.

In a further embodiment, the amount of methylphenidate in the dosage form is determined by the body weight of the patient using the following equation:

Dose (mg)=−0.00003*WT(lb)³+0.0081*WT(lb)²−0.123*WT(lb)+13.347±3.59

wherein WT is the weight in pounds of the patient and the calculated dose is in milligrams. The above equation provides a range of dosage amounts for a given body weight. To select the appropriate amount of methylphenidate from the range of dosage amounts for a patient of a particular weight, those skilled in the art will readily recognize the additional factors that can be considered by the prescribing physician when selecting the dosage amount. Such factors can include whether the patient is methylphenidate naïve or methylphenidate experienced, the age, gender or ethnicity of the patient, the severity of the patient's symptoms, and whether the patient suffers from any co-morbidities. In certain embodiments, the selection of dosage amount based on the body weight of the patient is for patients with body weight between about 20 lbs to about 150 lbs.

Patients that are currently being treated for ADHD with methylphenidate or other stimulant drugs, or are not receiving any stimulant treatment, can be treated in accordance with the methods of the present invention. Patients that have never received methylphenidate are considered methylphenidate naïve patients. Patients that have been treated with methylphenidate previously or are being treated with methylphenidate at the time the patients are administered the first dosage forms in accordance with the invention are considered methylphenidate experienced patients. Unlike some other methylphenidate treatment regimens, the methods and formulations of the present invention do not require a washout period prior to treating a patient in accordance with the present invention. Further, no conversion calculation is needed in order to switch a patient currently being treated with another formulation of methylphenidate to the method and formulations of the instant invention. In one embodiment, the patient in need of treatment is a methylphenidate naïve patient. In another embodiment, the patient in need of treatment is a methylphenidate experienced patient.

In certain embodiments, patients that are methylphenidate naïve, or patients that have other factors influencing the amount of methylphenidate that is prescribed, may receive an initial dosage amount that is less than the amount determined using the equation and/or tables above. In these patients, a target dose is determined using the tables or equation described above, but the patients can be administered an initial dosage amount that is lower than the target dosage amount or range of amounts determined by the tables or the equation above. The dosage amount administered to the patient is then titrated up to the target dose incrementally over a short period of time. For example, methylphenidate treatment can be initiated at low doses as a single daily dose in the morning. The initial dose may be in the range of 10 mg/day, up to 0.5 mg/kg/day, which under a physician's guidance may be increased in 10 mg increments after a minimum of 3 days, or 4 days, or at least weekly, to the target dose described above. In one embodiment, the initial dosage amount is at least 10 mg less than the target dosage amount. Thus, in certain embodiments of the invention is provided a method of reducing or eliminating a titration period of a treatment regimen for treating attention deficit hyperactivity disorder (ADHD) in a patient with methylphenidate comprising determining the body weight of the patient in need of treatment, determining the target dosage amount of methylphenidate to be administered to the patient, selecting a dosage form of a controlled-release formulation of methylphenidate to be administered to the patient containing a dosage amount that is less than the target dosage amount determined for such patient, and titrating the dosage amount up to the target dosage amount in increments until the target dosage amount is achieved, wherein the target dosage amount is determined by the body weight of the patient and correlates to the tables or equation presented above. The incremental increases can be selected from 5 mg, 10 mg and 15 mg. In some embodiments the incremental increases are the same. In other embodiments, the incremental increases are not the same.

In certain embodiments, the dosage amount selected based upon the patient's body weight is a target dosage amount and the patient's initial dosage amount is less than the target dosage amount. Therefore, in certain embodiments the present invention provides a method of treating attention deficit hyperactivity disorder in a patient comprising (a) determining the body weight of the patient in need of treatment, (b) selecting a target dose of methylphenidate to be administered to the patient wherein the target dose is selected based on the body weight of the patient, administering to the patient an initial dosage form comprising a dose of methylphenidate that is less than or equal to the target dose selected in step (b), and (c) administering to the patient a first subsequent dosage form comprising a dose of methylphenidate that is equal to the target dose selected in step (b) or is 5 mg or 10 mg greater than the dose in step (c) if the dose in step (c) is at least 5 mg or 10 mg less, respectively, than the target dose selected in step (b), wherein the body weight of the patient and the selected target dose correlate to the tables or equation presented above. In another embodiment, the method may further comprise (e) administering to the patient a second subsequent dosage form comprising a dose of methylphenidate that is equal to the target dose selected in step (b) or is 10 mg greater than the dose in step (d) if the dose in step (d) is at least 10 mg less than the target dose selected in step (b).

Further embodiments of the present invention provide a method of treating attention deficit hyperactivity disorder in a patient comprising (a) determining the body weight of the patient in need of treatment, (b) selecting a target dose of methylphenidate to be administered to the patient, wherein the target dose is selected based on the body weight of the patient, (c) administering to the patient once a day for a first seven day period of treatment an initial dosage form comprising a dose of methylphenidate that is less than the target dose selected in step (b), (d) administering to the patient once a day for a second seven day period of treatment a first subsequent dosage form comprising a dose of methylphenidate that is equal to the target dose selected in step (b) or is 5 mg or 10 mg greater than the dose in step (c) if the dose in step (c) is at least 5 mg or 10 mg less, respectively, than the target dose selected in step (b), (e) administering to the patient once a day for a third or greater seven day period of treatment a second or greater subsequent dosage form comprising a dose of methylphenidate that is equal to the target dose selected in step (b) or is 10 mg greater than the dose in the immediately prior seven day period if the dose in the immediately prior seven day period is at least 10 mg less than the target dose selected in step (b), and (f) repeating step (e) as often as needed until the dose of methylphenidate in the second or greater subsequent dosage form is equal to the target dose selected in step (b), wherein the body weight of the patient and the selected target dose correlate to one of the tables or the equation presented above.

In additional embodiments, the present invention provides a method of reducing or eliminating the titration period of a treatment regimen for treating attention deficit hyperactivity disorder in a patient comprising (a) determining the body weight of the patient in need of treatment, (b) selecting a target dose of methylphenidate to be administered to the patient wherein the target dose is selected based on the body weight of the patient, administering to the patient an initial dosage form comprising a dose of methylphenidate that is less than or equal to the target dose selected in step (b), and (c) administering to the patient a first subsequent dosage form comprising a dose of methylphenidate that is equal to the target dose selected in step (b) or is 5 mg or 10 mg greater than the dose in step (c) if the dose in step (c) is at least 5 mg or 10 mg less, respectively, than the target dose selected in step (b), wherein the body weight of the patient and the selected target dose correlate to the tables or equation presented above. In another embodiment, the method may further comprise (e) administering to the patient a second subsequent dosage form comprising a dose of methylphenidate that is equal to the target dose selected in step (b) or is 10 mg greater than the dose in step (d) if the dose in step (d) is at least 10 mg less than the target dose selected in step (b).

Further embodiments of the present invention provide a method of reducing or eliminating the titration period of a treatment regimen for treating attention deficit hyperactivity disorder in a patient comprising (a) determining the body weight of the patient in need of treatment, (b) selecting a target dose of methylphenidate to be administered to the patient, wherein the target dose is selected based on the body weight of the patient, (c) administering to the patient once a day for a first seven day period of treatment an initial dosage form comprising a dose of methylphenidate that is less than the target dose selected in step (b), (d) administering to the patient once a day for a second seven day period of treatment a first subsequent dosage form comprising a dose of methylphenidate that is equal to the target dose selected in step (b) or is 5 mg or 10 mg greater than the dose in step (c) if the dose in step (c) is at least 5 mg or 10 mg less, respectively, than the target dose selected in step (b), (e) administering to the patient once a day for a third or greater seven day period of treatment a second or greater subsequent dosage form comprising a dose of methylphenidate that is equal to the target dose selected in step (b) or is 10 mg greater than the dose in the immediately prior seven day period if the dose in the immediately prior seven day period is at least 10 mg less than the target dose selected in step (b), and (f) repeating step (e) as often as needed until the dose of methylphenidate in the second or greater subsequent dosage form is equal to the target dose selected in step (b), wherein the body weight of the patient and the selected target dose correlate to one of the tables or the equation presented above.

The following examples illustrate certain aspects and embodiments of the invention and are not to be construed as limiting the scope of the claims in any manner whatsoever. In addition, although embodiments of the invention may be described individually, the invention also encompasses any combination of two or more embodiments described herein.

### Examples

Study Formulations Administered in Clinical Studies:

Study Formulations were administered in the studies described below. Dosage strengths include total methylphenidate in the amounts of 10 mg, 15 mg, 20 mg, 30 mg, 40 mg, 50 mg, 60 mg, and 80 mg in accordance with Table 1.

TABLE 1

| Ingredient | % |
|---|---|
| Methylphenidate HCl (USP) | 12.78 |
| Sugar spheres 14/18 mesh (NF) | 62.02 |
| Opadry Clear YS-1-7006 (HS) | 4.04 |
| Methacrylic acid copolymer, Type B | 5.44 |
| (Eudragit RS 30 D) (NF) | |
| Methacrylic acid copolymer, Type C | 8.15 |
| (Eudragit L 30 D-55) (NF) | |
| Triethyl citrate (NF) | 2.70 |
| Talc (USP) | 4.87 |
| Purified water (USP)* | — |
| Colloidal Silicon Dioxide^{#} | — |
| Total Weight of Beads (mg) | 100.0 |

*Water is removed during manufacturing.
^{#}used as required and removed during processing

Study Formulations comprised multi-layer bead in capsule formulations, wherein the immediate release portion of methylphenidate is approximately 37% of the total methylphenidate in the dosage form and the remainder of methylphenidate is contained in the delayed release portion of the dosage form.

Adult Pharmacokinetic Studies

Single Dose Study in Adults:

This was a single-center, randomized, open-label, single-dose, three Period, crossover study to assess the relative bioavailability of Ritalin® Immediate Release tablets 3×25 mg given at 0, 4 and 8 hours, and Study Formulation 80 mg given as an oral dose (capsule or bead sprinkle) at 0 hour, in healthy adult male and female subjects. Twenty six (26) subjects were randomized to receive each of the three formulations during each of the periods during the study. During each Period, following an overnight fast of at least 10 hours, subjects received single oral doses of the Study Formulation 80 mg or Ritalin® Immediate Release tablets 3×25 mg given at 0, 4 and 8 hours. Serial blood samples for determination of methylphenidate plasma concentration and PK analysis were obtained on the day of dosing at time 0 (within 15 minutes pre-dose) and 0.5, 1, 1.5, 2, 2.5, 3, 4, 4.5, 5, 5.5, 6, 6.5, 7, 8, 8.5, 9, 9.5, 10, 10.5, 12, 15, 19 and 24 hours post-dose. Patients underwent a 7-day washout between each study Period.

Steady State Study in Adults:

This was a single-center, randomized, open-label, single- and multiple-dose, two Period, crossover study to assess the relative bioavailability of Ritalin® Immediate Release tablets 3×25 mg given at 0, 4 and 8 hours and Study Formulation 80 mg given at 0 hour as single doses under fed conditions and as multiple doses under fed conditions in healthy adult male and female subjects. Twenty six (26) subjects were randomized to receive both treatments in the study. During each Period, following an overnight fast of at least 10 hours, subjects completed a high fat breakfast approximately 5 minutes prior to dosing. Subjects then received single oral doses of Ritalin® Immediate Release tablets 3×25 mg given at 0, 4 and 8 hours or Study Formulation at time 0 with approximately 240 mL water. Subjects received their assigned doses during each Period on Day 2, Day 3 and Day 4. Serial blood samples for determination of methylphenidate plasma concentration and PK analysis were obtained each Period on Day 1 and Day 4 at time 0 (within 15 minutes pre-dose) and 0.5, 1, 1.5, 2, 2.5, 3, 4, 4.5, 5, 5.5, 6, 6.5, 7, 8, 8.5, 9, 9.5, 10, 10.5, 12, 15, 19 and 24 hours post-dose; on Day 2 and Day 3 at 4, 8, 12, 16 and 24 hours post-dose. Subjects were discharged from the research facility on Day 5. Patients underwent an 11-day washout prior to commencing the second study Period.

The final datasets for the single dose analysis contained approximately 1100 methylphenidate concentration measurements from approximately 25 subjects, and the multiple dose analysis contained approximately 1100 methylphenidate concentration measurements from approximately 20 subjects. Subjects receiving Ritalin® Immediate Release tablets were excluded from all modeling analyses, but were included in the AUC₀₋₄ calculation. In total, there were approximately 1600 methylphenidate concentration measurements from approximately 25 subjects for the pAUC₀₋₄ calculation from the single dose study.

Pediatric and Adolescent Efficacy Studies

Multi-Phase Pediatric and Adolescent Efficacy Study:

A parallel, randomized, double-blind, multicenter, placebo-controlled, forced dose, study was conducted to evaluate the safety and efficacy of Study Formulations in the treatment of ADHD in pediatric and adolescent patients aged 6 to 18 years. Subjects received their double-blind, randomized dose (10, 15, 20, or 40 mg methylphenidate or placebo) for 1 week. Following the Double-Blind phase, doses were optimized via titration in an open-label manner and subjects continued receiving methylphenidate for 11 weeks. Patients underwent screening assessments and a minimum 48-hour washout of previous stimulant medication if applicable. 221 subjects completed the Double-Blind phase and 200 subjects completed the Open-Label phase.

Double-Blind Phase:

During the Double-Blind phase, subjects participated in a Baseline Visit at Day 0, during which they underwent baseline assessments and were dispensed a randomized, double-blind, fixed dose of 10, 15, 20, and 40 mg/day Study Formulation or placebo for 1 week. The first dose of double-blind study drug was taken the morning of Day 1. Subjects returned 7 to 10 days following the Baseline visit for assessments.

Open-Label Phase:

The Open Label phase began immediately following the Double Blind phase. During the 11-week Open-Label phase, subjects received any of the available strengths of Study Formulation (i.e. 10, 15, 20, 30, 40, 50, and 60 mg). Subjects returned for evaluation weekly for 3 weeks, and then monthly for the remainder of the study. All patients received 10 mg doses of Study Formulations at the start of the Open-Label phase, and dose was optimised for each patient at 3 to 7 day increments until optimal dose was achieved.

Pediatric and Adolescent Pharmacokinetic Study

A single-center, randomized, open-label, two-way crossover study in 17 patients to compare the pharmacokinetics (PK) of Study Formulation (10, 15, 20, 30, and 40 mg strengths) and Ritalin® Immediate-Release Tablet (10 and 20 mg strengths) in young children with ADHD. At the time of study enrollment, all study patients were on some form of methylphenidate therapy. Patients aged 6 to 12 years were randomized to receive a total daily dose equivalent to the total daily methylphenidate dose that the patient was receiving at study enrollment. Prior to receiving methylphenidate as part of the study, patients stopped their current methylphenidate therapy and underwent a washout period for seven days. Following the washout period, patients in phase one received either as a single capsule of Study Formulation or two equal doses of Ritalin® IR. Serial blood samples were collected to determine methylphenidate plasma concentrations. PK analysis was obtained on the day of dosing at time 0 (pre-dose) and 1, 2, 3, 4, 5, 6, 8, 10, 12, and 24 hours post-dose. Following a 14 day break, patients in phase two received whichever formulation that they did not receive in phase one, as either a single capsule of Study Formulation or two equal doses of Ritalin® IR.

The final datasets for the pediatric population PK analysis contained approximately 150 methylphenidate concentration measurements from approximately 17 patients, and the pediatric population PK/PD analysis contained approximately 1700 change from baseline ADHD total scores measurements from approximately 270 patients.

### Example 1

### Pharmacokinetics Model in Adults

A base pharmacokinetics model (Base PK model) was established using the two-parallel input model with elimination from a single compartment, fit to the concentration-time data from the Single Dose Study in Adults using the first-order conditional estimation with interaction method in Phoenix NLME. The final model consists of 6 parameters: F1 represents the fraction of dose in the immediate release layer, Ka1 represents the first-order absorption rate for methylphenidate in the immediate release layer, Ka2 represents the first-order absorption rate for methylphenidate in the extended release layer, tlag represents the lag time for methylphenidate in the extended release layer, CL represents the apparent plasma clearance of methylphenidate, and V represents the apparent volume of distribution of methylphenidate. Between individual variability parameters were included on Ka1, Ka2, CL, and V. The proportional residual error model was superior to the additive error model and the combined (additive and proportional) error model. The data observed in the adult PK/PD studies described above was compared to the simulated data from the Base PK model for the equivalent Study Formulation dosage. The majority of the observed data was within the 5^{th} and 95^{th} percentiles of the simulated data, suggesting that the Base PK model was adequate for further simulations.

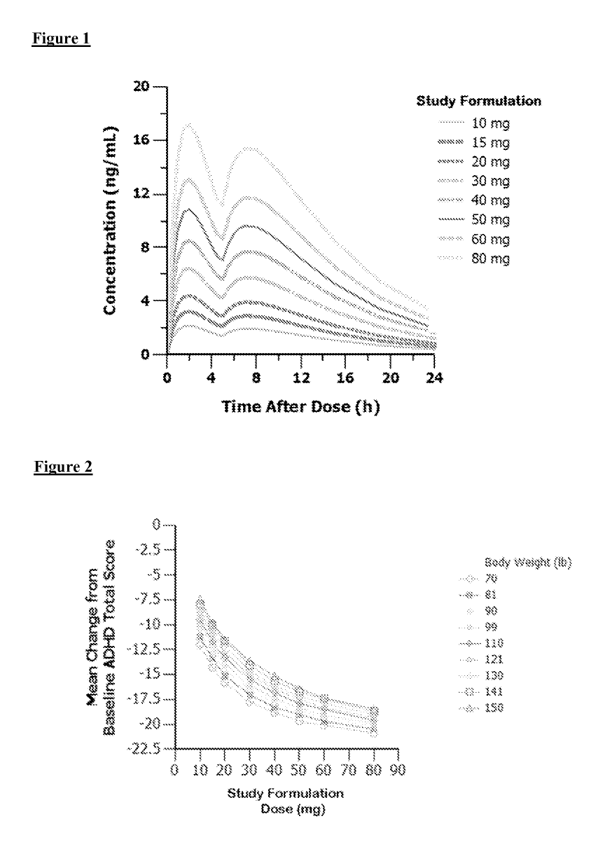

Simulations were performed to estimate the methylphenidate concentration-time profile after a single dose of Study Formulation at the following dose levels: 10 mg, 15 mg, 20 mg, 30 mg, 40 mg, 50 mg, 60 mg, and 80 mg. A single simulation was performed for each dose level with the final parameters from the Base PK model. Each simulation included approximately 100 replicates for the approximately 25 subjects from the Single Dose Adult Study for a total of approximately 2500 concentration-time profiles. The simulated data was further analyzed using Phoenix WinNonlin. The simulation data for each single dose and a 24-hour concentration time profile is represented in FIG. 1.

Considering the bimodal release profile of the Study Formulation, partial AUC at 0 to 4 hours was calculated for each dose level, again using the 80 mg dose of Study Formulation for a posterior predictive check. Results are shown in Table 2.

TABLE 2
Descriptive statistics for the overall mean pAUC₀₋₄ from
simulated methylphenidate concentration-time data.

| Biphentin ™ | Mean pAUC₀₋₄ | | |
|---|---|---|---|
| Dose (mg) | (ng*h/mL) | SD | % CV |
| 10 | 7.02 | 0.058 | 8.32 |
| 15 | 10.45 | 0.75 | 7.13 |
| 20 | 14.33 | 0.94 | 6.58 |
| 30 | 2100 | 1.46 | 6.95 |
| 40 | 27.69 | 2.07 | 7.49 |
| 50 | 35.26 | 2.72 | 7.72 |
| 60 | 42.66 | 3.10 | 7.27 |
| 80 | 55.84 | 3.98 | 7.12 |

### Example 2

### Pharmacokinetics Model in Pediatric Patients

A model similar to the Base PK model was established using the same two-input, 1-compartment, 1^{st} order elimination model based on the data collected from the Pediatric and Adolescent Pharmacokinetic Study. The pediatric PK model was consistent with the Base PK model. In addition, the potential effects of body weight, height and body mass index on the PK of methylphenidate in pediatric patients were evaluated using a stepwise addition and deletion method. Each covariate was evaluated for its potential impact on the volume of distribution, clearance, and lag time parameters of the population pharmacokinetic model. The final model included body weight as a covariate of methylphenidate clearance as shown in Equation 1:

CLi=CLTV*WTθe^{ηjCL} Equation 1:
where:
CLi=the true clearance for individual i
CLTV=the typical value (population mean) for clearance
WT=body weight for individual i
θ=Effect of body weight on clearance
ηiCL=the difference between the true value for individual i and the typical value for the population, with a mean of 0 and a variance of ω2.

### Pharmacokinetics/Pharmacodynamics Model in Pediatric Patients

The pediatric PK/PD model was developed using change from baseline ADHD total scores obtained from the Multi-Phase Pediatric and Adolescent Efficacy Study. The change from baseline ADHD total score response data was fit to an Eₘₐₓ model using the first-order conditional estimation with interaction method in Phoenix NLME. The dependent variable in the model was the change from baseline ADHD total score and the independent variable in the model was the simulated Cₘₐₓ for methylphenidate for each patient based on the final pediatric population PK model. The Eₘₐₓ model consists of 2 parameters: Eₘₐₓ represents the maximum change from baseline ADHD total score and EC50 represents the methylphenidate Cₘₐₓ required to achieve 50% of the Eₘₐₓ. Between individual variability parameters were included on Eₘₐₓ and EC50, and a proportional residual error model was used.

Monte Carlo simulations were performed with the final population PK/PD model for Study Formulation using the final parameter estimates for PK and PD parameters, between subject variability, and residual variability. Simulations were performed for patients with body weights ranging from 70 lb. to 150 lb. and Study Formulation dose strengths of 10 mg, 15 mg, 20 mg, 30 mg, 40 mg, 50 mg, 60 mg, and 80 mg. Each simulation combination (body weight and dose strength) was performed in replicates of 200 patients.

The effect of body weight on Study Formulation dose strength on clinical response (change from baseline ADHD total score) was evaluated by conducting multiple simulations. Body weight is inversely correlated with the maximum methylphenidate exposure such that as body weight increases at a given dose strength, the Cmax decreases. And Cmax is negatively correlated with the change from baseline ADHD total score such that as Cmax increases, the ADHD total score decreases further. These relationships are shown by plotting the mean change from baseline ADHD total score versus body weight using a single dose strength or the mean change from baseline ADHD total score versus dose strength using a single body weight.

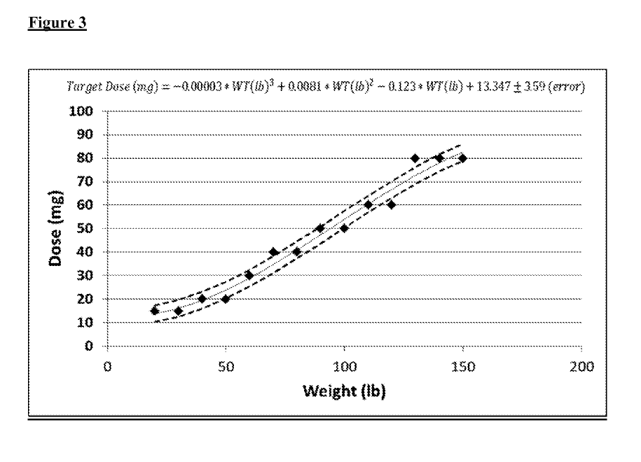

The simulated response profiles for body weight ranging from 70 lb. to 150 lb. across all Study Formulation dose strengths is shown in FIG. 2. In general, the response increases with increasing dose strength and decreasing body weight. Clinical management of symptoms requires a decrease in ADHD total score of at least 18 points. The Study Formulation dose strength required to achieve an 18 point reduction in ADHD total score for a range of body weights is shown in Table 3.

TABLE 3

| | | Mean Change |
|---|---|---|
| Body Weight | Biphentin ™ | from Baseline |
| (lb) | Dose (mg) | ADHD Score |
| 70 | 40 | −18.81 |
| 80 | 40 | −18.28 |
| 90 | 50 | −18.67 |
| 100 | 50 | −18.35 |
| 110 | 60 | −18.52 |
| 120 | 60 | −18.35 |
| 130 | 80 | −19.02 |
| 140 | 80 | −18.67 |
| 150 | 80 | −18.46 |

### Example 3

The dose needed to achieve an 18-point reduction in total ADHD score for a patient in need of treatment can be determined from the body weight of the patient in pounds. Based on the human adult and pediatric studies described above and the simulation in Example 2, the dose or range of doses required to achieve an 18 point target reduction in total ADHD score can be plotted against the body weight of the patient, and the data can be fit to a 3^{rd} order polynomial equation for each body weight measurement in pounds as shown in FIG. 3. The amount of methylphenidate in milligrams for a given patient body weight can be determined using the following equation:

Dose (mg)=−0.00003*WT (lb)³+0.0081*WT (lb)²−0.123*WT (lb)+13.347±3.59

wherein WT is the patient body weight in pounds.

For patients of particular weights, the ranges of dose amounts determined based on body weight are shown in Table 4. Such determinations are exemplary embodiments of the invention.

TABLE 4

| wt (lbs) | low (mg) | high (mg) |
|---|---|---|
| 30 | 13 | 20 |
| 40 | 16 | 23 |
| 50 | 20 | 27 |
| 60 | 25 | 32 |
| 70 | 31 | 38 |
| 80 | 36 | 44 |
| 90 | 42 | 50 |
| 100 | 48 | 56 |
| 110 | 54 | 61 |
| 120 | 60 | 67 |
| 130 | 65 | 72 |
| 140 | 69 | 76 |
| 150 | 72 | 79 |

### Example 4

The simulation completed in Example 2 is further applied to fixed methylphenidate doses in the range of 10 mg to 80 mg. The results are shown in Table 5 as the dosage amount of methylphenidate in a formulation according to the present invention to be administered to a patient of the corresponding body weight.

TABLE 5

| Patient Body | Amount of |
|---|---|
| Weight (lb) | Methylphenidate (mg) |
| ≦40 | 10-15 |
| 40-50 | 20 |
| 50-70 | 30 |
| 70-90 | 40 |
| 90-110 | 50 |
| 110-130 | 60 |
| ≧130 | 80 |

### Example 5. Fluctuation Index

Fluctuation index was calculated from the Single Dose and Steady State Adult Pharmacokinetic Studies described above, using the following formula:

FI=(C₍ₘₐₓ₎−C₍ₘᵢₙ₎)/C₍ₐᵥₑ₎

The FI is calculated for methylphenidate naïve subjects. Single dose pharmacokinetics are shown in Table 6. Steady state pharmacokinetics are shown in Table 7.

TABLE 6

| | Study Formulation | Study Formulation | Ritalin IR |
|---|---|---|---|
| | (Capsule) | (Sprinkle) | (3 × day) |
| 1^{st} Peak | 0.6277 | 0.4876 | 0.4027 |
| 2^{nd} Peak | 0.0423 | 0.0696 | 0.7861 |
| 3^{rd} Peak | N/A | N/A | 0.9181 |

TABLE 7

| | Study Formulation | Ritalin IR ® |
|---|---|---|
| | (Capsule) | (3 × day) |
| Day 1 | | |
| 1^{st} Peak | 0.1743 | 0.5757 |
| 2^{nd} Peak | 0.0114 | 0.5264 |
| 3^{rd} Peak | N/A | 0.7431 |
| Day 4 | | |
| 1^{st} Peak | 0.4635 | 0.6736 |
| 2^{nd} Peak | 0.0127 | 0.6523 |
| 3^{rd} Peak | N/A | 0.8540 |

1. A method of treating attention deficit hyperactivity disorder in a patient comprising:
a) determining the body weight of the patient in need of treatment;
b) selecting a dose of methylphenidate to be administered to the patient, wherein the dose is selected based on the body weight of the patient; and
c) administering a dosage form comprising the dose of step (b) to the patient, wherein the body weight of the patient and the selected dose correlate to
one of the following:

| Patient Body | | Dose of |
|---|---|---|
| Weight (lb) | | Methylphenidate (mg) |
| i) | ≦40 | 10 to 15 |
| ii) | 40-50 | 15 to 20 |
| iii) | 50-70 | 20 to 30 |
| iv) | 70-90 | 30 to 40 |
| v) | 90-110 | 40 to 50 |
| vi) | 110-130 | 50 to 60 |
| vii) | ≧130 | 60 to 80 |

2. The method of claim 1, wherein the body weight of the patient and the selected dose correlate to one of the following:

| Patient Body | | Dose of |
|---|---|---|
| Weight (lb) | | Methylphenidate (mg) |
| i) | ≦40 | 10 to 15 |
| ii) | 40-50 | 15 to 20 |
| iii) | 50-70 | 20 to 30 |
| iv) | 70-90 | 30 to 40 |
| v) | 90-110 | 40 to 50 |
| vi) | 110-130 | 50 to 60 |

3. The method of claim 1, wherein the dosage form comprises methylphenidate hydrochloride.

4. The method of claim 1, wherein the dosage form provides a controlled release of the methylphenidate.

5. The method of claim 4, wherein the controlled release dosage form provides (i) an immediate release portion of the methylphenidate and (ii) a delayed release portion of the methylphenidate.

6. The method of claim 4, wherein the controlled release dosage form is administered once daily.

7. The method of claim 4, wherein the dose of methylphenidate in the dosage form is selected from the group consisting of 10 mg, 15 mg, 20 mg, 30 mg, 40 mg, 50 mg, 60 mg and 80 mg.

8. The method of claim 1, wherein the dosage form is administered to the patient as an initial dose, or as a subsequent dose, or as a target dose.

9-10. (canceled)

11. The method of claim 4, wherein the controlled release dosage form provides a time to maximum plasma concentration at about 0.5 to about 4 hours after oral administration and wherein the duration of effect falls below an effective plasma concentration at about 8 to about 12 hours after oral administration.

12. The method of claim 1, wherein the method decreases a total ADHD-RS-IV score in the patient by a value of 18 or more.

13. The method of claim 1, wherein the body weight of the patient is between about 20 lb and about 150 lb.

14. The method of claim 1, wherein the patient is treated for a period of 2 days or more.

15. The method of claim 1, wherein the patient is treated for a period of 10 days or more.

16. The method of claim 1, wherein the patient is treated for a period of 30 days or more.

17. A method of treating attention deficit hyperactivity disorder in a patient comprising:
a. determining the body weight of the patient in need of treatment;
b. selecting a target dose of methylphenidate to be administered to the patient, wherein the target dose is selected based on the body weight of the patient;
c. administering to the patient an initial dosage form comprising a dose of methylphenidate that is less than or equal to the target dose selected in step (b); and
d. administering to the patient a first subsequent dosage form comprising a dose of methylphenidate that is equal to the target dose selected in step (b) or is 5 mg or 10 mg greater than the dose in step (c) if the dose in step (c) is at least 5 mg or 10 mg less, respectively, than the target dose selected in step (b),
wherein the body weight of the patient and the selected target dose correlate to one of the following:

| Patient Body | | Target Dose of |
|---|---|---|
| Weight (lb) | | Methylphenidate (mg) |
| i) | ≦40 | 10 to 15 |
| ii) | 40-50 | 15 to 20 |
| iii) | 50-70 | 20 to 30 |
| iv) | 70-90 | 30 to 40 |
| v) | 90-110 | 40 to 50 |
| vi) | 110-130 | 50 to 60 |
| vii) | ≧130 | 60 o 80 |

18. The method of claim 17, further comprising:
e. administering to the patient a second subsequent dosage form comprising a dose of methylphenidate that is equal to the target dose selected in step (b) or is 10 mg greater than the dose in step (d) if the dose in step (d) is at least 10 mg less than the target dose selected in step (b).

19. The method of claim 17 or 18, wherein the body weight of the patient and the selected target dose correlate to one of the following:

| Patient Body | | Target Dose of |
|---|---|---|
| Weight (lb) | | Methylphenidate (mg) |
| i) | ≦40 | 10 to 15 |
| ii) | 40-50 | 15 to 20 |
| iii) | 50-70 | 20 to 30 |
| iv) | 70-90 | 30 to 40 |
| v) | 90-110 | 40 to 50 |
| vi) | 110-130 | 50 o 60 |

20. The method of claim 17, wherein the initial and subsequent dosage forms comprise methylphenidate hydrochloride.

21. The method of claim 17, wherein the initial and subsequent dosage forms provide a controlled release of the methylphenidate.

22. The method of claim 21, wherein the controlled release dosage forms provide (i) an immediate release portion of the methylphenidate and (ii) a delayed release portion of the methylphenidate.

23. The method of claim 21, wherein the controlled release dosage forms are administered once daily.

24. The method of claim 21, wherein the dose of methylphenidate in the dosage forms is selected from the group consisting of 10 mg, 15 mg, 20 mg, 30 mg, 40 mg, 50 mg, 60 mg and 80 mg.

25. The method of claim 21, wherein the controlled release dosage forms provide a time to maximum plasma concentration at about 0.5 to about 4 hours after oral administration and wherein the duration of effect falls below an effective plasma concentration at about 8 to about 12 hours after oral administration.

26. The method of claim 17, wherein the method decreases a total ADHD-RS-IV score in the patient by a value of 18 or more.

27. The method of claim 17, wherein the body weight of the patient is between about 20 lb and about 150 lb.

28. The method of claim 17, wherein the initial dosage form is administered for at least 4 days.

29. The method of claim 28, wherein the initial dosage form is administered for at least 7 days.

30. The method of claim 17, wherein the first subsequent dosage form is administered for at least 4 days.

31. The method of claim 30, wherein the first subsequent dosage form is administered for at least 7 days.

32. The method of claim 18, wherein the second subsequent dosage form is administered for at least 4 days.

33. The method of claim 32, wherein the second subsequent dosage form is administered for at least 7 days.

34. The method of claim 17, wherein the initial dosage form comprises a 10 mg dose of methylphenidate.

35. The method of claim 17, wherein the initial dosage form comprises a dose of methylphenidate that is at least 10 mg less than the target dose of methylphenidate selected in step (b).

36. A method of treating attention deficit hyperactivity disorder in a patient comprising:
a. determining the body weight of the patient in need of treatment;
b. selecting a target dose of methylphenidate to be administered to the patient, wherein the target dose is selected based on the body weight of the patient;
c. administering to the patient once a day for a first seven day period of treatment an initial dosage form comprising a dose of methylphenidate that is less than the target dose selected in step (b);
d. administering to the patient once a day for a second seven day period of treatment a first subsequent dosage form comprising a dose of methylphenidate that is equal to the target dose selected in step (b) or is 5 mg or 10 mg greater than the dose in step (c) if the dose in step (c) is at least 5 mg or 10 mg less, respectively, than the target dose selected in step (b);
e. administering to the patient once a day for a third or greater seven day period of treatment a second or greater subsequent dosage form comprising a dose of methylphenidate that is equal to the target dose selected in step (b) or is 10 mg greater than the dose in the immediately prior seven day period if the dose in the immediately prior seven day period is at least 10 mg less than the target dose selected in step (b); and
f. repeating step (e) as often as needed until the dose of methylphenidate in the second or greater subsequent dosage form is equal to the target dose selected in step (b),
wherein the body weight of the patient and the selected target dose correlate to one of the following:

| Patient Body | | Target Dose of |
|---|---|---|
| Weight (lb) | | Methylphenidate (mg) |
| i) | ≦40 | 10 to 15 |
| ii) | 40-50 | 15 to 20 |
| iii) | 50-70 | 20 to 30 |
| iv) | 70-90 | 30 to 40 |
| v) | 90-110 | 40 to 50 |
| vi) | 110-130 | 50 to 60 |
| vii) | ≧130 | 60 o 80 |

37-38. (canceled)

39. A method of reducing or eliminating the titration period of a treatment regimen for treating attention deficit hyperactivity disorder with methylphenidate in a patient, comprising:
g. determining the body weight of the patient in need of treatment;
h. selecting a target dose of methylphenidate to be administered to the patient, wherein the target dose is selected based on the body weight of the patient;
i. administering to the patient once a day for a first seven day period of treatment an initial dosage form comprising a dose of methylphenidate that is less than the target dose selected in step (b);
j. administering to the patient once a day for a second seven day period of treatment a first subsequent dosage form comprising a dose of methylphenidate that is equal to the target dose selected in step (b) or is 5 mg or 10 mg greater than the dose in step (c) if the dose in step (c) is at least 5 mg or 10 mg less, respectively, than the target dose selected in step (b);
k. administering to the patient once a day for a third or greater seven day period of treatment a second or greater subsequent dosage form comprising a dose of methylphenidate that is equal to the target dose selected in step (b) or is 10 mg greater than the dose in the immediately prior seven day period of treatment if the dose in the immediately prior seven day period of treatment is at least 10 mg less than the target dose selected in step (b); and
l. repeating step (e) as often as needed until the dose of methylphenidate in the second or greater subsequent dosage form is equal to the target dose selected in step (b),
wherein the body weight of the patient and the selected target dose correlate to one of the following:

| Patient Body | | Target Dose of |
|---|---|---|
| Weight (lb) | | Methylphenidate (mg) |
| i) | ≦40 | 10 to 15 |
| ii) | 40-50 | 15 to 20 |
| iii) | 50-70 | 20 to 30 |
| iv) | 70-90 | 30 to 40 |
| v) | 90-110 | 40 to 50 |
| vi) | 110-130 | 50 to 60 |
| vii) | ≧130 | 60 o 80 |

40. (canceled)
